(19) United States
(12) Patent Application Publication
(10) Pub. No.: US 2009/0326496 A1
(43) Pub. Date: Dec. 31, 2009

(54) TRANSPARENT HYDROGEL WOUND DRESSING

(75) Inventors: Andrew David Court, Cheshire (GB); June Michaela Gladman, Cheshire (GB)

Correspondence Address: CONVATEC INC., 100 HEADQUARTERS PARK DRIVE, SKILLMAN, NJ, 08558, US

(73) Assignee: BRISTOL-MYERS SQUIBB COMPANY, New York, NY (US)

(21) Appl. No.: 11/720,912

(22) PCT Filed: Dec. 6, 2005
(86) PCT No.: PCT/GB2005/004687

§ 371 Date: Aug. 4, 2009

(30) Foreign Application Priority Data
Dec. 6, 2004 (GB) 0426718.3

(51) Int. Cl. A61F 13/53

(52) U.S. Cl. 604/368

(57) ABSTRACT

A wound dressing comprising a hydrogel polymer in sheet form reinforced with a fabric comprising gel-forming fibres which fabric is capable of absorbing exudate to allow the wound to be viewed through the dressing.

This invention relates to a hydrogel sheet dressing which is suitable for use as a wound dressing. More particularly, this invention relates to a hydrogel sheet dressing reinforced with a fibrous material.

Various types of hydrogel sheet dressings are known and are available commercially. WO 95/26699 describes a composite material for wound dressings having a fibrous wound-contacting substrate, such as cotton gauze, impregnated with a thermoplastic hydrogel-forming polymer. The hydrogel is said to be present to prevent the gauze from sticking to the wound and provide some absorptive capacity to the dressing.

Hydrogels can be described generally as insoluble polymers with hydrophilic sites which absorb and interact with significant volumes of liquid, particularly water or in the case of wound dressings, wound exudate. They are generally of two types: amorphous hydrogels which progressively decrease in viscosity as they absorb exudate until the gel eventually loses all its cohesive properties and effectively becomes a dispersion of the polymer in water; and hydrogels which have a stable macro structure which do not change their physical form as they absorb fluid, although they may swell and increase in volume. This swelling process continues until the gel becomes fully saturated or until equilibrium is reached. When hydrogels are used on wounds it is generally necessary to include a reinforcement to prevent the swollen or less viscous hydrogel from disintegrating upon removal from the wound.

In the past, hydrogels have been reinforced with cotton gauze or a close spaced polymer net. This type of reinforcement prevents or impedes the visualisation of the wound through the dressing. Many medical practitioners wish to be able to visualise the wound through the dressing so that the state of the wound can be assessed without disrupting the healing process taking place under the dressing. Known reinforcements for hydrogels impair or obscure the view so that this cannot be easily achieved. In addition to this, known reinforcements for hydrogels do little to increase the absorptive capacity of the dressing above that of the hydrogel itself.

Suprisingly, we have found that hydrogels can be reinforced with fabrics made from woven or non-woven gel-forming fibres. Such reinforcing materials become transparent or translucent on absorption of exudate allowing visualisation of the wound through the dressing. Such reinforcing materials are also capable of absorbing exudate in their own right and thus contribute to the total absorptive capacity of the dressing while performing the function of allowing the dressing to be removed from the wound in one piece.

Accordingly, the present invention provides a wound dressing comprising a hydrogel polymer in sheet form reinforced with a fabric comprising gel-forming fibres, which fabric is capable of absorbing exudate to allow the wound to be viewed through the dressing.

Accordingly, another aspect of the present invention provides a wound dressing comprising a hydrogel polymer in sheet form reinforced with a fabric comprising absorbent fibres.

We have found that wound dressings according to the invention may mitigate the problems associated with the viewing of wounds yet allow the hydrogel to be removed from the wound in one piece. It is thought that this is in part achieved by the selection of the reinforcing fabric which provides absorbency and allows the wound to be viewed on the absorption of exudate. This allows the wound to be viewed without removal of the dressing.

The reinforcing fabric comprises gel-forming fibres and the fabric is capable of absorbing exudate to allow the wound to be viewed through the dressing.

The gel-forming fibres are preferably chemically modified cellulosic fibres in the form of a fabric and in particular carboxymethylated cellulose fabrics as described in EP 0616650 or EP 0680344 or EP 1091770. The carboxymethylated cellulosic fabrics preferably have a degree of substitution of between 0.12 to 0.35 as measured by IR spectroscopy (as defined in WO/00/01425) and are made by carboxymethylating a woven or non-woven cellulosic fabric such that the absorbency is increased. Particularly preferred fabrics have an absorbency of between 10 g/g of sodium/calcium chloride as defined above to 30 g/g of sodium/calcium chloride as measured by the method defined above. Particularly preferred fabrics have an absorbency of 15 g/g to 30 g/g and most preferred of 20 g/g to 28 g/g of sodium/calcium chloride as measured by the method defined above.

The cellulosic fabric preferably consists solely of cellulosic fibre but may contain a proportion of non-cellulosic textile fibre or of gel-forming fibre. The cellulosic fibre is of known kind and may comprise continuous filament yarn and/or staple fibre. The carboxymethylation is generally performed by contacting the fabric with an alkali and a carboxymethylating agent such as chloracetic acid in an aqueous system.

The fabric is preferably of a non-woven type to reduce fibre shedding in the wound on cutting of the dressing.

Other types of gel-forming fibres may be used, in particular fibres for use in the present invention are preferably hygroscopic fibres which upon the uptake of wound exudate become moist and slippery and allow the wound to be seen through the dressing. The gel-forming fibres may be fibres of alginate, viscose, modified cellulose, cellulose, polyester, polypropylene and co-polymers thereof, pectin, chitosan fibres, hyaluronic acid fibres or other polysaccharide fibres or fibres derived from gums. Preferably the gel-forming fibres for use in the present invention have an absorbency of at least 15 g/g of water as measured by the 1996 British Pharmacopoeia free swell test, more preferably between 25 g/g and 60 g/g. Most preferred are highly absorbent gel-forming fibres such as modified cellulose fibres as described above.

The hydrogel used in the dressing of the present invention may be an A-B-A block copolymer, multiblock copolymer, graft copolymer or polymer blend, each incorporating a hydrophilic component and a hydrophobic component. The hydrogel polymer may be water-insoluble yet water-swellable and highly absorbent.

Preferably, the hydrogel layer comprises a hydrogel material selected from polyurethane gels, biopolymer gels, carboxymethyl cellulose gels, hydroxyethyl cellulose gels, hydroxy propyl methyl cellulose, modified acrylamide and mixtures thereof. Suitable biopolymer gels include alginates, pectins, galactomannans, chitosan, gelatin, hyaluronates and mixtures thereof. Preferably, the gels are cross-linked, and the cross-linking may be either covalent or ionic.

Preferably, the hydrogel material further comprises from 5 to 50% by weight on a dry weight basis of one or more humectants such as glycerol.

The hydrogel may be a polyacrylamide and, in particular, a derivatised polyacrylamide copolymer containing a sodium sulphonated group, glycerol and water. The hydrogel may also be a polyacrylate. An example of a suitable polyacrylamide is a graft copolymer of N,N,dimethylacrylamide and a polystyrene-based macromonomer prepared by free radical initiated solution copolymerisation. The starting materials are reacted in the presence of a polymerisation solvent, such as ethyl acetate, ethanol, methyl ethyl acetone, acetone, tetrahydrofuran, mixtures thereof and the like and a polymerisation catalyst (e.g. asobisisobutyronitrile at a reaction temperature in the range of up to about 80° C. The resulting solution containing the copolymer is then optionally purified to remove unreacted monomer and other impurities. For example, the copolymer solution may be precipitated with a non-solvent such as an ether compound. The resulting precipitated copolymer is separated and dried.

The hydrogel polymer may be melt processed through an extruder to impregnate the hydrogel onto the fabric reinforcement. The dressing is then compressed to form a sheet dressing. Alternatively, the reinforced hydrogels of the invention may be made by coating or impregnating a continuous web of the fabric reinforcement with hydrogel monomers and then reacting the monomers to form the hydrogel. In this way the hydrogel forms an intimate contact with the fibres to entrap the fabric.

The wound dressing of the present invention can absorb wound fluid without being dissolved away from the wound site. The hydrogel polymer allows the wound to be visualised through the dressing and, through careful choice of the reinforcing fabric, that also allows the wound to be seen.

1. A wound dressing comprising a hydrogel polymer in sheet form reinforced with a fabric comprising gel-forming fibres which fabric is capable of absorbing exudate to allow the wound to be viewed through the dressing.

2. A wound dressing as claimed in claim 1 characterised in that the gel-forming fibres are chemically modified cellulosic fibres.

3. A wound dressing as claimed in claim 2 characterised in that the reinforcing fabric is a carboxymethylated cellulose fabric having a degree of substitution of between 0.12 to 0.35 as measured by IR spectroscopy.

4. A wound dressing comprising a hydrogel polymer in sheet form reinforced with a fabric comprising absorbent fibres.

5. A wound dressing as claimed in claim 4 characterised in that the absorbent fibres are gel-forming fibres.

6. A wound dressing as claimed in claim 5 characterised in that the gel-forming fibres are chemically modified cellulosic fibres

7. A method of treating a wound comprising applying to the wound a wound dressing of claim 1.

8. A wound dressing as claimed in claim 1 characterized in that the hydrogel is selected from polyurethane gels, biopolymer gels, carboxymethyl cellulose gels, hydroxyethyl cellulose gels, hydroxyl propyl methyl cellulose, modified acrylamide and mixtures thereof.

9. A wound dressing as claimed in claim 4 characterized in that the hydrogel is selected from polyurethane gels, biopolymer gels, carboxymethyl cellulose gels, hydroxyethyl cellulose gels, hydroxyl propyl methyl cellulose, modified acrylamide and mixtures thereof.
